SEQUENCE LISTING (20 sequences)

SEQ ID NO: 1 - protein, 578 aa, Punctularia strigosozonata
  REGION 1..578: note = alpha 1,4-glucosidase (glucoamylase) Pstr_GA_0048
MLSSLIVSGLLASGVCASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEVASSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDVFYDGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 2 - DNA, 1753 bp, Punctularia strigosozonata
  misc_feature 1..1753: note = alpha 1,4-glucosidase (glucoamylase) Pstr_GA_0048
  source 1..1753: mol_type = genomic DNA
ggtctcgaatgttgtcctctttgatcgtttccggtttgttggcttctggtgtctgtgcctcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaagttgcctcttcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagatgttttctacgacggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaaggagacc

SEQ ID NO: 3 - protein, 580 aa, Punctularia strigosozonata
  REGION 1..580: note = alpha 1,4-glucosidase (glucoamylase) Pstr_GA_0009
MRFPSIFTAVLFAASSALASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEINSSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDVFMGGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 4 - DNA, 1759 bp, Punctularia strigosozonata
  misc_feature 1..1759: note = alpha 1,4-glucosidase (glucoamylase) Pstr_GA_0009
  source 1..1759: mol_type = genomic DNA
ggtctcgaatgagattcccatccatcttcaccgccgtcttgttcgctgcttcttctgctttggcttcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaaatcaactcctcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagatgttttcatgggtggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaaggagacc

SEQ ID NO: 5 - protein, 580 aa, Punctularia strigosozonata
  REGION 1..580: note = alpha 1,4-glucosidase (glucoamylase) Pstr_GA_0010
MRFPSIFTAVLFAASSALASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEINSSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDSFMGGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 6 - DNA, 1759 bp, Punctularia strigosozonata
  misc_feature 1..1759: note = alpha 1,4-glucosidase (glucoamylase) Pstr_GA_0010
  source 1..1759: mol_type = genomic DNA
ggtctcgaatgagattcccatccatcttcaccgccgtcttgttcgctgcttcttctgctttggcttcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaaatcaactcctcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagattctttcatgggtggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaaggagacc

SEQ ID NO: 7 - protein, 580 aa, Punctularia strigosozonata
  REGION 1..580: note = alpha 1,4-glucosidase (glucoamylase) Pstr_GA_0033
MRFPSIFTAVLFAASSALASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEVASSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDVFMGGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 8 - DNA, 1759 bp, Punctularia strigosozonata
  misc_feature 1..1759: note = alpha 1,4-glucosidase (glucoamylase) Pstr_GA_0033
  source 1..1759: mol_type = genomic DNA
ggtctcgaatgagattcccatccatcttcaccgccgtcttgttcgctgcttcttctgctttggcttcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaagttgcctcttcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagatgttttcatgggtggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaaggagacc

SEQ ID NO: 9 - protein, 580 aa, Punctularia strigosozonata
  REGION 1..580: note = alpha 1,4-glucosidase (glucoamylase) Pstr_GA_0034
MRFPSIFTAVLFAASSALASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEVASSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDSFMGGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 10 - DNA, 1759 bp, Punctularia strigosozonata
  misc_feature 1..1759: note = alpha 1,4-glucosidase (glucoamylase) Pstr_GA_0034
  source 1..1759: mol_type = genomic DNA
ggtctcgaatgagattcccatccatcttcaccgccgtcttgttcgctgcttcttctgctttggcttcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaagttgcctcttcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagattctttcatgggtggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaaggagacc

SEQ ID NO: 11 - DNA, 57 bp, synthetic construct
  misc_feature 1..57: note = S. cerevisiae MATalpha signal sequence
  source 1..57: mol_type = other DNA
atgagattcccatccatcttcaccgccgtcttgttcgctgcttcttctgctttggct

SEQ ID NO: 12 - DNA, 621 bp, synthetic construct
  misc_feature 1..621: note = Fragment containing the PGK1-promoter
  source 1..621: mol_type = other DNA
ggtctcggtgcgggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgggagacc

SEQ ID NO: 13 - DNA, 322 bp, synthetic construct
  misc_feature 1..322: note = Fragment containing ENO1 terminator
  source 1..322: mol_type = other DNA
ggtctcgtaaaagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcccctcggagacc

SEQ ID NO: 14 - DNA, 50 bp, synthetic construct
  misc_feature 1..50: note = Connector 2L
  source 1..50: mol_type = other DNA
tctgcaccttctttcgcttgcactgttcccagactccaagatttgtatac

SEQ ID NO: 15 - DNA, 50 bp, synthetic construct
  misc_feature 1..50: note = Connector 2M
  source 1..50: mol_type = other DNA
ccaatacaaggcagaactttcctccaccgtatccgctcgcaatttctttc

SEQ ID NO: 16 - DNA, 150 bp, synthetic construct
  misc_feature 1..150: note = Sc_INT28_FLANK5
  source 1..150: mol_type = other DNA
ttatttcttttttacagaaaaatgaaagcaatagaattgaatttcaacaagtcacaaacaaactttgtagactttttattacaaaaatttattaagtgactctgcaccttctttcgcttgcactgttcccagactccaagatttgtatac

SEQ ID NO: 17 - DNA, 150 bp, synthetic construct
  misc_feature 1..150: note = Sc_INT28_FLANK3
  source 1..150: mol_type = other DNA
ccaatacaaggcagaactttcctccaccgtatccgctcgcaatttctttccattttcctcatgcgggacgccagctttggtgcgggatgaaggcaatatccaaaccagcatcaggaaaagcctgaagaaatgtggtgcctaatgctctta

SEQ ID NO: 18 - DNA, 20 bp, synthetic construct
  misc_feature 1..20: note = INT28 protospacer
  source 1..20: mol_type = other DNA
gagcagttctcagattacaa

SEQ ID NO: 19 - DNA, 21 bp, synthetic construct
  misc_feature 1..21: note = Primer
  source 1..21: mol_type = other DNA
tggatgtgtgtgcagtatgct

SEQ ID NO: 20 - DNA, 24 bp, synthetic construct
  misc_feature 1..24: note = Primer
  source 1..24: mol_type = other DNA
acaggaagtcgagcgtgtctgggt

(19) United States
(12) Patent Application Publication
(10) Pub. No.: US 2024/0409965 A1
(43) Pub. Date: Dec. 12, 2024

(54) VARIANT POLYPEPTIDE AND RECOMBINANT YEAST CELL

(71) Applicant: DANISCO US INC., Palo Alto, CA (US)

(72) Inventors: Hans Marinus Charles Johannes DE BRUIJN, Echt (NL); Evert Tjeerd VAN RIJ, Echt (NL); Mickel Leonardus August JANSEN, Echt (NL); Marco Richard VAN DER WEERT, Echt (NL); Wouter KROES, Echt (NL); Johannes Gustaaf Ernst VAN LEEUWEN, Echt (NL)

(21) Appl. No.: 18/704,733

(22) PCT Filed: Nov. 4, 2022
(86) PCT No.: PCT/EP2022/080763

§ 371 Date: Apr. 25, 2024

(30) Foreign Application Priority Data
Nov. 4, 2021 (EP) 21206524.7

(51) Int. Cl. C12P 7/06; C12N 1/18; C12N 9/34; C12R 1/865

(52) U.S. Cl. CPC C12P 7/06; C12N 1/18; C12N 9/2428; C12R 2001/865; C12Y 302/01003

(57) ABSTRACT

A variant polypeptide of a parent polypeptide, wherein the parent polypeptide comprises the amino acid sequence of SEQ ID NO: 1, and wherein the variant polypeptide comprises an amino acid sequence which, when aligned with the amino acid sequence of SEQ ID NO: 1, comprises an amino acid substitution of V202I, A203N, V333S, Y335M and/or D336G, the positions of said amino acids being defined with reference to the amino acid sequence of SEQ ID NO: 1. A recombinant yeast cell functionally expressing a nucleotide encoding the variant polypeptide and a process for the production of ethanol using the variant polypeptide and/or recombinant yeast.

### FIELD OF THE INVENTION

The invention relates to a novel variant polypeptide, a novel recombinant yeast cell and a process for producing ethanol wherein said variant polypeptide or novel recombinant yeast cell is used.

### BACKGROUND OF THE INVENTION

Microbial fermentation processes from renewable carbohydrate feedstocks are applied in the industrial production of a broad and rapidly expanding range of chemical compounds. Ethanol production by Saccharomyces cerevisiae is currently, by volume, the single largest fermentation process in industrial biotechnology. Various approaches have been proposed to improve the fermentative properties of organisms used in industrial biotechnology by genetic modification.

In literature several different approaches have been reported for ethanol production from starch-containing material.

Traditionally a multi-step process is applied, including both enzymatic hydrolysis and yeast-based fermentation. As a first step, amylase and glucoamylase enzyme can be added to the starch-containing media to produce glucose. The glucose can be converted in a yeast-based fermentation to ethanol. For example, US2017/0306310 describes a process of producing a fermentation product, particularly ethanol, from starch-containing material comprising the steps of: (a) liquefying starch-containing material in the presence of an alpha amylase; (b) saccharifying the liquefied material; and (c) fermenting with a fermenting organism; wherein step (b) is carried out using at least a variant glucoamylase. U.S. Pat. No. 10,227,613 describes a process for producing fermentation products from starch-containing material comprising the steps of i) liquefying the starch-containing material using an alpha-amylase in the presence of a protease; ii) saccharifying the liquefied starch-containing material using a carbohydrate-source generating enzyme; and iii) fermenting using a fermenting organism, wherein a cellulolytic composition comprising two or more enzymes selected from the group consisting of an endoglucanase, a beta-glucosidase, a cellobiohydrolase, and a polypeptide having cellulolytic enhancing activity is present or added during fermentation or simultaneous saccharification and fermentation.

Alternatively, yeast can be transformed with a glucoamylase gene. WO2019063543A1 and WO 2020/043497 describe an especially active glucoamylase and useful yeasts expressing such glucoamylase. For example, WO 2020/043497 describes a process for the production of ethanol comprising fermenting a corn slurry under anaerobic conditions in the presence of a recombinant yeast; and recovering the ethanol, wherein said recombinant yeast functionally expresses a heterologous nucleic acid sequence encoding a certain glucoamylase, wherein the process comprises dosing a glucoamylase at a concentration of 0.05 g/L or less.

Although good results are obtained with the above yeasts and processes, further improvement remains desirable.

It would be an advancement in the art to provide a yeast producing glucoamylase with an even further increased activity.

### SUMMARY OF THE INVENTION

The inventors have now found a way to further increase the activity of the glucoamylase described in WO2019063543A1 and WO 2020/043497, advantageously allowing for a recombinant yeast cell and a process for the production of ethanol using such recombinant yeast cell, where di-saccharides, oligo-saccharides and/or polysaccharides, comprising mono-saccharides linked to each other via an alpha-1,4-glycosidic bond, can be hydrolysed quicker and even more efficiently.

Accordingly the present invention provides a variant polypeptide of a parent polypeptide, wherein the parent polypeptide comprises the amino acid sequence of SEQ ID NO: 1, and wherein the variant polypeptide comprises an amino acid sequence which, when aligned with the amino acid sequence of SEQ ID NO: 1, comprises an amino acid substitution of V202I (Valine 202 Isoleucine) and/or A203N (Alanine 203 Asparagine) and/or V333S (Valine 333 Serine) and/or Y335M (Tyrosine 335 Methionine) and/or D336G (Aspartic acid 336 Glycine), the positions of said amino acids being defined with reference to the amino acid sequence of SEQ ID NO: 1.

The invention further provides a polypeptide comprising or consisting of an amino acid sequence of SEQ ID NO:03, SEQ ID NO:05, SEQ ID NO: 07 or SEQ ID NO:09 or an amino acid sequence having equal to or more than 70%, and more preferably equal to or more than 75%, 80%, 85%, 90%, 95, 98%, or 99%, sequence identity with the amino acid sequence of SEQ ID NO:03, SEQ ID NO:05, SEQ ID NO: 07 and/or SEQ ID NO:09.

Still further the invention provides a nucleotide sequence comprising or consisting of nucleotide sequence of respectively SEQ ID NO:04, SEQ ID NO:06, SEQ ID NO: 08 or SEQ ID NO:10.

The invention also provides a recombinant yeast cell functionally expressing a nucleotide sequence encoding the above variant polypeptide or the above nucleotide sequence.

Thus, the invention also provides a recombinant yeast cell comprising or functionally expressing a nucleotide sequence encoding a polypeptide, which polypeptide comprises or consists of an amino acid sequence of SEQ ID NO:03, SEQ ID NO:05, SEQ ID NO: 07 or SEQ ID NO:09 or an amino acid sequence having equal to or more than 70%, and more preferably equal to or more than 75%, 80%, 85%, 90%, 95, 98%, or 99%, sequence identity with the amino acid sequence of SEQ ID NO:03, SEQ ID NO: 05, SEQ ID NO: 07 and/or SEQ ID NO:09.

Finally the invention provides a process for the production of ethanol, comprising converting a carbon source, preferably a carbohydrate, using a polypeptide as described above or a recombinant yeast cell as described above.

As illustrated in the examples, the above aspects of the invention advantageously allows one to hydrolyse di-saccharides, oligo-saccharides or polysaccharides comprising an alpha-1,4-glycosidic bond, such as maltose, quicker and more efficiently. This in turn allows one to advantageously reduce or even avoid the amount of external, ex-situ produced, glucoamylase that needs to be added.

Use of the above peptide, recombinant yeast cell and/or the above process can thus advantageously result in reduction of total sugar content at the end of fermentation and/or could advantageously allow one to reduce or even refrain from dosing of glucoamylase during the fermentation.

That is, the use of the recombinant yeast cell according to the invention advantageously enables one to reduce the dosing of ex-situ produced or other external glucoamylase to the process by 10 to 100% whilst still allowing one to have the same total residual sugar content at the end of fermentation. In the alternative or in addition the use of the recombinant yeast cell according to the invention allows one to have a lower residual sugar content at the end of fermentation whilst adding the same low amount (or even no) external glucoamylase.

### BRIEF DESCRIPTION OF THE SEQUENCE LISTING

This application contains a Sequence Listing in computer readable form, which is incorporated herein by reference. An overview is provided by Table 1 below.

TABLE 1
Overview of sequence listings:

| SEQ ID | | | |
|---|---|---|---|
| No: | Enzyme Name | Origin | Type |
| SEQ ID | alpha-1,4-glucosidase | Punctularia | protein |
| NO: 01 | (glucoamylase, Pstr_GA_0048) | strigosozonata | |
| SEQ ID | alpha-1,4-glucosidase | Punctularia | DNA |
| NO: 02 | (glucoamylase, Pstr_GA_0048) | strigosozonata | |
| SEQ ID | alpha-1,4-glucosidase | Punctularia | protein |
| NO: 03 | (glucoamylase, Pstr_GA_0009) | strigosozonata | |
| SEQ ID | alpha-1,4-glucosidase | Punctularia | DNA |
| NO: 04 | (glucoamylase, Pstr_GA_0009) | strigosozonata | |
| SEQ ID | alpha-1,4-glucosidase | Punctularia | protein |
| NO: 05 | (glucoamylase, Pstr_GA_0010) | strigosozonata | |
| SEQ ID | alpha-1,4-glucosidase | Punctularia | DNA |
| NO: 06 | (glucoamylase, Pstr_GA_0010) | strigosozonata | |
| SEQ ID | alpha-1,4-glucosidase | Punctularia | protein |
| NO: 07 | (glucoamylase, Pstr_GA_0033) | strigosozonata | |
| SEQ ID | alpha-1,4-glucosidase | Punctularia | DNA |
| NO: 08 | (glucoamylase, Pstr_GA_0033) | strigosozonata | |
| SEQ ID | alpha-1,4-glucosidase | Punctularia | protein |
| NO: 09 | (glucoamylase, Pstr_GA_0034) | strigosozonata | |
| SEQ ID | alpha-1,4-glucosidase | Punctularia | DNA |
| NO: 10 | (glucoamylase, Pstr_GA_0034) | strigosozonata | |
| SEQ ID | S. cerevisiae MATalpha | Artificial | DNA |
| NO: 11 | signal sequence | Sequence | |
| SEQ ID | Fragment containing the | Artificial | DNA |
| NO: 12 | S. cerevisiae | Sequence | |
| | PGK1-promoter (Sc_PGK1.pro) | | |
| SEQ ID | Fragment containing the | Artificial | DNA |
| NO: 13 | S. cerevisiae | Sequence | |
| | ENO1-terminator (Sc_ENO1.ter) | | |
| SEQ ID | Connector 2L | Artificial | DNA |
| NO: 14 | | Sequence | |
| SEQ ID | Connector 2M | Artificial | DNA |
| NO: 15 | | Sequence | |
| SEQ ID | Sc_INT_028_FLANK5 | Artificial | DNA |
| NO: 16 | | Sequence | |
| SEQ ID | Sc_INT_028_FLANK3 | Artificial | DNA |
| NO: 17 | | Sequence | |
| SEQ ID | INT_028 protospacer | Artificial | DNA |
| NO: 18 | | Sequence | |
| SEQ ID | Primer | Artificial | DNA |
| NO: 19 | | Sequence | |
| SEQ ID | Primer | Artificial | DNA |
| NO: 20 | | Sequence | |

### DEFINITIONS

Unless defined otherwise or clearly indicated by context, all technical and scientific terms used herein have the same meaning as commonly understood by one of ordinary skill in the art.

Unless explicitly indicated otherwise, the various embodiments of the invention described herein can be cross-combined.

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows. The articles “a” and “an” are used herein to refer to one or to more than one (i.e. to one or at least one) of the grammatical object of the article. By way of example, “an amino acid” may mean one amino acid or more than one amino acid.

Amino acid sequence aligned with the amino acid sequence set out in SEQ ID NO: X (when referring to a variant polypeptide) means that the variant amino acid sequence and the amino acid sequence set out in SEQ ID NO: X are aligned by a suitable method which allows comparison of the sequences with each other and identifications of the positions in the amino acid sequence of the variant wherein either the same amino acid is present (identical position), or another amino acid is present (substitution), or one or more extra amino acids are present (insertion or extension) or no amino acid is present (deletion or truncation) if compared with the amino acid sequence set out in SEQ ID NO: X.

A suitable method allowing comparison of two amino acid sequence may be any suitable Pairwise Sequence Alignment method known to those skilled in the art, preferably a Global Pairwise Sequence Alignment method. A preferred Global Pairwise Sequence Alignment method is the EMBOSS Needle method based on the Needleman-Wunsch alignment algorithm (aiming at finding the optimum alignment (including gaps) of the two sequences along their entire length) (Needleman, S. B. and Wunsch, C. D. (1970) J. Mol. Biol. 48, 443-453) as described herein. In one embodiment the amino acid sequence is aligned with the amino acid sequence set out in SEQ ID NO: x using the NEEDLE program from the EMBOSS package, using EBLOSUM62 as a substitution matrix, with a gap-open penalty of 10 and a gap extension penalty of 0.5.

A “yeast” or “yeast cell” as defined herein is a yeast suitable for genetic manipulation and which may be cultured at cell densities useful for industrial production of a target product. A yeast or yeast cell may be found in nature or a cell derived from a parent cell after genetic manipulation or classical mutagenesis.

The term “control sequence” as used herein refers to components involved in the regulation of the expression of a coding sequence in a specific organism or in vitro. Examples of control sequences are transcription initiation sequences, termination sequences, promoters, leaders, signal peptides, propeptides, prepropeptides, or enhancer sequences; Shine-Delgarno sequences, repressor or activator sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion.

The term “culturing” refers to a method of multiplying microorganisms in a nutrient medium and under conditions suitable for the growth and/or propagation of said microorganism and/or the production of a compound of interest by the microorganism. These methods are known in the art. When the microorganism is able to express/produce a compound of interest, for example, the microorganisms may be cultured by shake flask culturing, small-scale or large-scale fermentation (including continuous, batch, fed-batch, or solid state fermentations) in laboratory or industrial fermenters performed in a suitable medium and under conditions allowing the compound of interest to be expressed and/or isolated. Typically, the culturing will comprise a growth phase mainly directed to formation of biomass and a production phase mainly directed to production of the compound of interest. The growth phase and production phase may overlap to some extent. A suitable nutrient medium comprises carbon sources, nitrogen sources and additional compounds (such as inorganic salts (e.g. phosphate), trace elements and/or vitamins) (see, e. g., Bennett, J. W. and LaSure, L., eds., More Gene Manipulations in Fungi, Academic Press, CA, 1991) and can be performed under aerobic or anaerobic conditions.

The term “derived from” also includes the terms “originates from,” “obtained from,” “obtainable from,” “isolated from,” and “created from,” and typically indicates that one specified material finds its origin in another specified material or has features that can be described with reference to the another specified material. As used herein, a substance (e.g., a nucleic acid molecule or polypeptide) “derived from” a microorganism preferably means that the substance is native to that microorganism.

The term “expression” includes any step involved in the production of (a) polypeptide(s) including, but not limited to, transcription, post transcriptional modification, translation, post-translational modification, and secretion. A reduction or abolishment of production of B means a limitation to x % or less B produced via enzymatic conversion of A. This can be achieved with an enzyme/protein/cell/gene as described herein. An increase in production of B means an increase of at least x % B produced via enzymatic conversion of A compared to the amount B obtained in a process using a non-modified cell/wild type protein/enzyme/gene. Reduction or increase of gene expression can be measured by various methods, such as e.g. Northern, Southern or Western blot technology as known in the art. The terms “increase of activity” or “overexpression” are used interchangeably herein.

An expression cassette comprises a polynucleotide coding for a polypeptide, operably linked to the appropriate control sequences which allow for expression of the polynucleotide in a cell or in vitro.

The expression cassette may be an autonomously replicating vector (e.g plasmid), i. e., a vector the replication of which is independent of genome replication. Alternatively, the cassette may be one which, when introduced into the cell, is fully or partially integrated into the genome of the cell. In the latter cases it may comprise one or more targeting sequences to direct integration into the genome.

The expression cassette may or may not contain one or more selectable markers, which permit easy selection of transformed cells.

The term “polypeptide fragment” is defined herein as a polypeptide having one or more amino acids deleted from the amino and/or carboxyl terminus of the parent polypeptide.

The term “mature polypeptide” is defined herein as a polypeptide in its final form(s) and is obtained after translation of a mRNA into polypeptide, post-translational modifications of said polypeptide in or outside the cell. Post-translational modification include N-terminal processing, C-terminal truncation, glycosylation, phosphorylation and removal of leader sequences such as signal peptides, propeptides and/or prepropeptides as defined herein by cleavage.

The term “naturally-occurring” as used herein refers to processes, events, or products that occur in their relevant form in nature. By contrast, “not naturally-occurring” refers to processes, events, or products whose existence or form involves the hand of man. The term “non-naturally occurring is herein synomymous with “synthetic”. Generally, the term “naturally-occurring” with regard to polypeptides or nucleic acids can be used interchangeable with the term “wild-type” or “native”. It refers to polypeptide or nucleic acids encoding a polypeptide, having an amino acid sequence or polynucleotide sequence, respectively, identical to that found in nature. Naturally occurring polypeptides include native polypeptides, such as those polypeptides naturally expressed or found in a particular cell. Naturally occurring polynucleotides include native polynucleotides such as those polynucleotides naturally found in the genome of a particular cell. Additionally, a sequence that is wild-type or naturally-occurring may refer to a sequence from which a variant or a synthetic sequence is derived.

The term “nucleic acid construct” is herein referred to as a nucleic acid molecule, either single- or double-stranded, which is derived from a naturally occurring gene or which has been modified to contain segments of nucleic acid which are combined and juxtaposed in a manner which would not otherwise exist in nature. Nucleic acid constructs can be isolated, synthetically made of mutagenized. The term nucleic acid construct is synonymous with the term “expression cassette” when the nucleic acid construct contains all the control sequences required for expression of a coding sequence, wherein said control sequences are operably linked to said coding sequence.

The term “operably linked” as used herein refers to two or more components such as nucleic acid sequences or polypeptide sequences that are physically linked and are in a functional relationship with each other permitting them to function in their intended manner. For instance, a promoter is operably linked to a coding sequence if the promoter can regulate the transcription or expression of a coding sequence, in which case the coding sequence should be understood as being “under the control of” the promoter.

The term “parent polypeptide” refers to the polypeptide relative to which another polypeptide differs by substituting, adding or deleting one or more amino acids.

Position being defined with reference to SEQ ID NO: x means that the position in the amino acid sequence according to the disclosure at which a modification has taken place is given in respect with the position of the corresponding amino acid in the amino acid sequence according to SEQ ID NO: x when the two sequences are aligned using an alignment method as described herein.

The term “recombinant” when used in reference to a nucleic acid, or protein indicates that the nucleic acid, or protein has been modified in its sequence if compared to its native form by human intervention. The term “recombinant” when referring to a cell indicates that the genome of the cell has been modified in its sequence if compared to its native form by human intervention. The term “recombinant” is synonymous with “genetically modified”.

A selectable marker is a gene which allows for selection of cells transformed with such a gene and which provides for biocide or viral resistance, resistance to heavy metals, prototrophy to auxotrophs, and the like.

Preferred selectable markers include, but are not limited to, those which confer resistance to drugs or which complement a defect in the cell. They include e. g. versatile marker genes that can be used for transformation of most filamentous fungi and yeasts such as acetamidase genes or cDNAs, or genes providing resistance to antibiotics.

Alternatively, specific selection markers can be used such as auxotrophic markers which require corresponding mutant strains. Preferably, the selection marker is deleted from the transformed cell after introduction of the expression construct so as to obtain transformed cells which are free of selection marker genes.

The term selectable marker extends to a marker gene used for screening, i.e. marker gene that, once introduced into a cell confers to the cell a visible phenotype and causes the cell look different. An example of marker for screening is a gene coding for a fluorescent protein which causes cells to fluoresce under an appropriate light source.

For the purpose of this disclosure, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this disclosure the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the disclosure is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

The nucleic acid and protein sequences of the present disclosure can further be used as a “query sequence” to perform a search against public databases to, for example, identify other family members or related sequences. Such searches can be performed using the BLASTN and BLASTX programs (version 2.0) of Altschul, et al. (1990) J. Mol. Biol. 215:403-10. BLAST nucleotide searches can be performed with the BLASTN program, score=100, wordlength=12 to obtain nucleotide sequences homologous to nucleic acid molecules of the disclosure. BLAST protein searches can be performed with the BLASTX program, score=50, wordlength=3 to obtain amino acid sequences homologous to protein molecules of the disclosure. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., BLASTX and BLASTN) can be used. See the homepage of the National Center for Biotechnology Information at http://www.ncbi.nlm.nih.gov/.

As used herein, the terms “variant, “derivative”, “mutant” or “homologue” can be used interchangeably. They can refer to either polypeptides or nucleic acids. Variants include substitutions, insertions, deletions, truncations, transversions, and/or inversions, at one or more locations relative to a reference sequence. Variants can be made for example by site-saturation mutagenesis, scanning mutagenesis, insertional mutagenesis, random mutagenesis, site-directed mutagenesis, and directed-evolution, as well as various other recombination approaches. Variant polypeptides may differ from a reference polypeptide by a small number of amino acid residues and may be defined by their level of primary amino acid sequence homology/identity with a reference polypeptide. Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a reference polypeptide. Methods for determining percent identity are known in the art and described herein. Generally, the variants retain the characteristic nature of the reference polypeptide, but have altered properties in some specific aspects. For example, a variant may have a modified pH optimum, a modified substrate binding ability, a modified resistance to enzymatic degradation or other degradation, an increased or decreased activity, a modified temperature or oxidative stability, but retains its characteristic functionality. Variants further include polypeptides with chemical modifications that change the characteristics of a reference polypeptide.

With regard to nucleic acids, the terms refer to a nucleic acid that encodes a variant polypeptide, that has a specified degree of homology/identity with a reference nucleic acid, or that hybridizes under stringent conditions to a reference nucleic acid or the complement thereof. Preferably, a variant nucleic acid has at least 70%, at least 75%, at least 80%, at least 85%, at least 90%, at least 91%, at least 92%, at least 93%, at least 94%, at least 95%, at least 96%, at least 97%, at least 98%, or even at least 99% nucleic acid sequence identity with a reference nucleic acid. Methods for determining percent identity are known in the art and described herein.

The term “encoding” has the same meaning as “coding for”. Thus, by way of example, “one or more genes encoding a glucoamylase” has the same meaning as “one or more genes coding for a glucoamylase”.

As far as genes or nucleic acid sequences encoding a protein or an enzyme are concerned, the phrase “one or more nucleic acid sequences encoding a X”, wherein X denotes a protein, has the same meaning as “one or more nucleic acid sequences encoding a protein having X activity”. Thus, by way of example, “one or more nucleic acid sequences encoding a glucoamylase” has the same meaning as “one or more nucleic acid sequences encoding a protein having glucoamylase activity”.

### The Variant Polypeptide

The polypeptide having amino acid sequence SEQ ID NO: 01 is preferably a polypeptide having glucoamylase activity. Such a polypeptide having glucoamylase activity is herein also referred to as simply “glucoamylase”.

Glucoamylase (EC 3.2.1.20 or 3.2.1.3), is also referred to as alpha 1,4-glucosidase, amyloglucosidase, alpha-glucosidase, glucan 1,4-alpha glucosidase, maltase glucoamylase, and maltase-glucoamylase, and such terms are used interchangeably herein. Glucoamylase can catalyse at least the hydrolysis of 1,4-linked alpha-D-glucose residues from non-reducing ends of amylose chains to release free D-glucose. Suitably the protein may have other or further activities. Preferably, however, the glucoamylase activity is dominating.

The polypeptide having amino acid sequence SEQ ID NO: 01 can suitable by encoded by a nucleotide sequence of SEQ ID NO: 02.

The variant polypeptide is preferably a variant of the polypeptide comprising or consisting of the amino acid sequence of SEQ ID NO: 1, wherein the variant comprises an amino acid sequence which, when aligned with the amino acid sequence of SEQ ID NO: 1, comprises an amino acid substitution of Y335M and/or D336G, optionally in combination with an amino acid substitution V202I, A203N and/or V333S, the positions of said amino acids being defined with reference to the amino acid sequence of SEQ ID NO: 1.

Preferably the variant polypeptide comprises or consists of an amino acid sequence having equal to or more than 70%, and more preferably equal to or more than 75%, 80%, 85%, 90%, 95, 98%, or 99%, sequence identity with the amino acid sequence of SEQ ID NO: 01.

More preferably any further amino acid substitutions in the variant polypeptide as compared to the parent polypeptide of SEQ ID NO: 1, that is any amino acid substitutions other than Y335M and/or D336G and/or V202I and/or A203N and/or V333S, are conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. In an embodiment, conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

More preferably the variant polypeptide is a polypeptide comprising or consisting of an amino acid sequence of SEQ ID NO:03, SEQ ID NO:05, SEQ ID NO: 07 or SEQ ID NO:09 or an amino acid sequence having equal to or more than 70%, and more preferably equal to or more than 75%, 80%, 85%, 90%, 95, 98%, or 99%, sequence identity with the amino acid sequence of SEQ ID NO:03, SEQ ID NO: 05, SEQ ID NO: 07 and/or SEQ ID NO: 09. Preferably any amino acid substitutions in the variant polypeptide as compared to the amino acid sequence of SEQ ID NO:03, SEQ ID NO:05, SEQ ID NO: 07 and/or SEQ ID NO:09 are conservative amino acid substitutions. Preferences and examples of such conservative amino acid substitutions are as described above.

The variant polypeptide is preferably a synthetic polypeptide.

The invention further provides a nucleic acid sequence encoding the variant polypeptide of the invention, preferably wherein said nucleic acid sequence is synthetic.

The polypeptides comprising or consisting of an amino acid sequence of respectively SEQ ID NO: 03, SEQ ID NO: 05, SEQ ID NO: 07 or SEQ ID NO: 09 can suitable by encoded by a nucleotide sequence comprising or consisting of nucleotide sequence of respectively SEQ ID NO:04, SEQ ID NO: 06, SEQ ID NO: 08 or SEQ ID NO:10.

The invention therefore also provides a nucleotide sequence comprising or consisting of nucleotide sequence of respectively SEQ ID NO:04, SEQ ID NO:06, SEQ ID NO: 08 or SEQ ID NO:10.

### The Recombinant Yeast Cell

The invention also provides a recombinant yeast cell which cell produces the variant polypeptide. The invention therefore also provides a recombinant yeast cell comprising or functionally expressing a nucleotide sequence encoding a polypeptide, which polypeptide comprises or consists of an amino acid sequence of SEQ ID NO:03, SEQ ID NO:05, SEQ ID NO: 07 or SEQ ID NO:09 or an amino acid sequence having equal to or more than 70%, and more preferably equal to or more than 75%, 80%, 85%, 90%, 95, 98%, or 99%, sequence identity with the amino acid sequence of SEQ ID NO: 03, SEQ ID NO:05, SEQ ID NO: 07 and/or SEQ ID NO:09.

Suitably such a recombinant yeast cell can be a recombinant yeast cell comprising or functionally expressing a nucleotide sequence comprising or consisting of nucleotide sequence SEQ ID NO: 04, SEQ ID NO:06, SEQ ID NO: 08 or SEQ ID NO:10 or a nucleotide sequence having equal to or more than 70%, and more preferably equal to or more than 75%, 80%, 85%, 90%, 95, 98%, or 99%, sequence identity with the nucleotide sequence of SEQ ID NO:04, SEQ ID NO:06, SEQ ID NO: 08 or SEQ ID NO:10.

Preferably the recombinant yeast cell is preferably a yeast cell, or derived from a yeast cell, from the genus of Saccharomycesceae or the genus of Schizosaccharomycesceae. That is, preferably the host cell from which the recombinant yeast cell is derived is a yeast cell from the genus of Saccharomycesceae or the genus of Schizosaccharomycesceae.

Examples of suitable yeast cells include Saccharomyces, such as Saccharomyces cerevisiae, Saccharomyces eubayanus, Saccharomyces jurei, Saccharomyces pastorianus, Saccharomyces beticus, Saccharomyces fermentati, Saccharomyces paradoxus, Saccharomyces uvarum and Saccharomyces bayanus.

Examples of suitable yeast cells further include Schizosaccharomyces, such as Schizosaccharomyces pombe, Schizosaccharomyces japonicus, Schizosaccharomyces octosporus and Schizosaccharomyces cryophilus.

Other exemplary yeasts include Torulaspora such as Torulaspora delbrueckii; Kluyveromyces such as Kluyveromyces marxianus; Pichia such as Pichia stipitis, Pichia pastoris or Pichia angusta; Zygosaccharomyces such as Zygosaccharomyces bailii; Brettanomyces such as Brettanomyces inter medius; Brettanomyces bruxellensis, Brettanomyces anomalus, Brettanomyces custersianus, Brettanomyces naardenensis, Brettanomyces nanus, Dekkera bruxellensis and Dekkera anomala; Metschmkowia, Issatchenkia, such as Issatchenkia orientalis, Kloeckera such as Kloeckera apiculata; and Aureobasidium such as Aureobasidium pullulans.

The yeast cell is preferably a yeast cell of the genus Schizosaccharomyces, herein also referred to as a Schizosaccharomyces yeast cell, or a yeast cell of the genus Saccharomyces, herein also referred to as a Saccharomyces yeast cell. More preferably the yeast cell is a yeast cell derived from a yeast cell of the species Saccharomyces cerevisiae, herein also referred to as a Saccharomyces cerevisae yeast cell. That is, preferably the host cell from which the recombinant yeast cell is derived is a yeast cell from the species Saccharomyces cerevisiae. Hence, preferably the recombinant yeast cell is a recombinant Saccharomyces cerevisiae yeast cell.

Preferably the yeast cell is an industrial yeast cell. The living environments of yeast cells in industrial processes are significantly different from that in the laboratory. Industrial yeast cells must be able to perform well under multiple environmental conditions which may vary during the process. Such variations include changes in nutrient sources, pH, ethanol concentration, temperature, oxygen concentration, etc., which together have potential impact on the cellular growth and ethanol production of the yeast cell. An industrial yeast cell can be understood to refer to a yeast cell that, when compared to a laboratory counterpart, has a more robust performance. That is, when compared to a laboratory counterpart, the industrial yeast cell shows less variation in performance when one or more environmental conditions selected from the group of nutrient sources, pH, ethanol concentration, temperature, oxygen concentration, are varied during fermentation. Preferably, the yeast cell is constructed on the basis of an industrial yeast cell as a host, wherein the construction is conducted as described hereinafter. Examples of industrial yeast cells are Ethanol Red® (Fermentis) Fermiol® (DSM) and Thermosacc® (Lallemand).

The recombinant yeast cell described herein may be derived from any host cell capable of producing a fermentation product. Preferably the host cell is a yeast cell, more preferably an industrial yeast cell as described herein above. Preferably the yeast cell described herein is derived from a host cell having the ability to produce ethanol.

The yeast cell described herein may be derived from the host cell through any technique known by one skilled in the art to be suitable therefore. Such techniques may include any one or more of mutagenesis, recombinant DNA technology (including, but not limited to, CRISPR-CAS techniques), selective and/or adaptive evolution, mating, cell fusion, and/or cytoduction between yeast strains. Suitably the one or more desired genes are incorporated in the yeast cell by a combination of one or more of the above techniques.

The recombinant yeast cells according to the invention are preferably inhibitor tolerant, i.e. they can withstand common inhibitors at the level that they typically have with common pretreatment and hydrolysis conditions, so that the recombinant yeast cells can find broad application, i.e. it has high applicability for different feedstock, different pretreatment methods and different hydrolysis conditions. In an embodiment the recombinant yeast cell is inhibitor tolerant. Inhibitor tolerance is resistance to inhibiting compounds. The presence and level of inhibitory compounds in lignocellulose may vary widely with variation of feedstock, pretreatment method hydrolysis process. Examples of categories of inhibitors are carboxylic acids, furans and/or phenolic compounds. Examples of carboxylic acids are lactic acid, acetic acid or formic acid. Examples of furans are furfural and hydroxy-methylfurfural. Examples or phenolic compounds are vannilin, syringic acid, ferulic acid and coumaric acid. The typical amounts of inhibitors are for carboxylic acids: several grams per liter, up to 20 grams per liter or more, depending on the feedstock, the pretreatment and the hydrolysis conditions. For furans: several hundreds of milligrams per liter up to several grams per liter, depending on the feedstock, the pretreatment and the hydrolysis conditions. For phenolics: several tens of milligrams per liter, up to a gram per liter, depending on the feedstock, the pretreatment and the hydrolysis conditions.

In an embodiment, the recombinant yeast cell is a cell that is naturally capable of alcoholic fermentation, preferably, anaerobic alcoholic fermentation. A recombinant yeast cell preferably has a high tolerance to ethanol, a high tolerance to low pH (i.e. capable of growth at a pH lower than about 5, about 4, about 3, or about 2.5) and towards organic and/or a high tolerance to elevated temperatures.

The recombinant yeast cell may comprise one, two, or more copies of a nucleotide sequence encoding the variant polypeptide. Suitably the recombinant yeast cell can comprise in the range from equal to or more than 1, preferably equal to or more than 2 to equal to or less than 30, preferably equal to or less than 20 and most preferably equal to or less than 10 copies of the nucleotide sequence encoding the variant polypeptide. Most preferably the recombinant yeast cell may comprise one, two, three, four, five, six, seven, eight, nine, ten, eleven or twelve copies of the nucleotide sequence encoding the the variant polypeptide.

A signal sequence (also referred to as signal peptide, targeting signal, localization signal, localization sequence, transit peptide, leader sequence or leader peptide) can be present at the N-terminus of a polypeptide (here, the GA) where it signals that the polypeptide is to be excreted, for example outside the cell and into the media.

Preferably the nucleotide sequence(s) encoding the variant polypeptide is codon optimized and any native signal sequences are replaced by those of the host cell. As indicated above, recombinant yeast host cells from the species Saccharomyces cerevisiae are preferred. Therefore, preferably the nucleotide sequence encoding the glucoamylase is codon optimized and any native signal sequences are replaced by the S. cerevisiae MATalpha signal sequence, more preferably the S. cerevisiae MATalpha signal nucleotide sequence of SEQ ID NO: 11.

The recombinant yeast may be subjected to evolutionary engineering to improve its properties. Evolutionary engineering processes are known processes. Evolutionary engineering is a process wherein industrially relevant phenotypes of a microorganism, herein the recombinant yeast, can be coupled to the specific growth rate and/or the affinity for a nutrient, by a process of rationally set-up natural selection. Evolutionary Engineering is for instance described in detail in Kuijper, M, et al, FEMS, Eukaryotic cell Research 5(2005) 925-934, WO2008041840 and WO2009112472. After the evolutionary engineering the resulting pentose fermenting recombinant cell is isolated. The isolation may be executed in any known manner, e.g. by separation of cells from a recombinant cell broth used in the evolutionary engineering, for instance by taking a cell sample or by filtration or centrifugation.

In an embodiment, the recombinant yeast is marker-free. As used herein, the term “marker” refers to a gene encoding a trait or a phenotype which permits the selection of, or the screening for, a host cell containing the marker. Marker-free means that markers are essentially absent in the recombinant yeast. Being marker-free is particularly advantageous when antibiotic markers have been used in construction of the recombinant yeast and are removed thereafter. Removal of markers may be done using any suitable prior art technique, e.g. intramolecular recombination.

In one embodiment, the recombinant yeast is constructed on the basis of an inhibitor tolerant host cell, wherein the construction is conducted as described hereinafter. Inhibitor tolerant host cells may be selected by screening strains for growth on inhibitors containing materials, such as illustrated in Kadar et al, Appl. Biochem. Biotechnol. (2007), Vol. 136-140, 847-858, wherein an inhibitor tolerant S. cerevisiae strain ATCC 26602 was selected.

In a preferred embodiment, the activity of the variant polypeptide described above is fine-tuned or upregulated by overexpression. Preferably the nucleotide sequence encoding the variant polypeptide is preceded by a promoter. The promoter can be a native promoter, a heterologous promoter or a synthetic promoter. Preferably the recombinant yeast cell is a recombinant Saccharomyces cerevisiae yeast cell and preferably the promoter is a promoter that is native to Saccharomyces cerevisiae.

More preferably the promoter is selected from the list consisting of: pTDH3, pPGK1, pHTA1, pTEF1, pPGK1, pPRS3, pYKT6, pACT1, pZOU1, pMYO4 and pPFY1, or a functional homologue thereof comprising a nucleotide sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity therewith. The promoter advantageously enables higher expression of the variant polypeptide, preferably by a multiplication factor of 2 or more.

In addition to the variant polypeptide according to the invention, the recombinant yeast cell can advantageously comprise or functionally express nucleotide sequences encoding for further polypeptides, respectively proteins, respectively enzymes. For example, the recombinant yeast cell is preferably a recombinant yeast cell further comprising or further functionally expressing functionally expressing: a nucleotide sequence encoding a protein having alpha-1,6-glucosidase activity and/or a nucleotide sequence encoding a protein having alpha-1,1-glucosidase activity and/or a nucleotide sequence encoding a protein having beta-glucosidase activity.

Advantageously the recombinant yeast cell can further comprise or functionally express:
a nucleotide sequence encoding a protein comprising phosphoketolase activity (EC 4.1.2.9 or EC 4.1.2.22); and/or
a nucleotide sequence encoding a protein having phosphotransacetylase (PTA) activity (EC 2.3.1.8); and/or
a nucleotide sequence encoding a protein having acetate kinase (ACK) activity (EC 2.7.2.12); and/or
a nucleotide sequence encoding a protein having ribulose-1,5-biphosphate carboxylase oxygenase (Rubisco) activity; and/or
a nucleotide sequence encoding a protein having phosphoribulokinase (PRK) activity; and/or
a nucleotide sequence encoding a protein comprising NADH dependent acetylating acetaldehyde dehydrogenase activity; and/or
a nucleotide sequence encoding a protein comprising acetyl-CoA synthetase activity; and/or
a nucleotide sequence encoding a protein comprising alcohol dehydrogenase activity; and/or
a nucleotide sequence encoding a protein having glycerol dehydrogenase activity (E.C. 1.1.1.6); and/or
a nucleotide sequence encoding a protein having dihydroxyacetone kinase activity (E.C. 2.7.1.28 or E.C. 2.7.1.29); and/or
a nucleotide sequence encoding a protein having glycerol transporter activity.
   Further preferences are detailed herein below.

### Dosing of External Glucoamylase

As indicated above, preferably the variant polypeptide is a polypeptide having glucoamylase activity. The in-situ production of such a variant polypeptide by a recombinant yeast according to the invention therefore advantageously allows one to reduce or even avoid the dosing of external, suitably ex-situ produced, glucoamylase. By the term “dosing” is herein understood the ex-situ addition of (external) glucoamylase, i.e. glucoamylase that is not in-situ produced by the yeast during the fermentation. Such external glucoamylase can be added, in addition to the glucoamylase that is already produced in-situ by the yeast that is functionally expressing glucoamylase.

For example, ex-situ produced glucoamylase can be dosed at a concentration between 0.005 and 0.05 g/L (gram per liter), between 0.01 and 0.05 g/L, between 0.02 and 0.05 g/L, between 0.03 and 0.05 g/L, or between 0.04 and 0.05 g/L. In an embodiment ex-situ produced glucoamylase is dosed at concentration between 0.005 and 0.04 g/L, between 0.01 and 0.04 g/L, between 0.02 and 0.04 g/L, or between 0.03 and 0.04 g/L. In an embodiment ex-situ produced glucoamylase is dosed at concentration between 0.005 and 0.04 g/L, between 0.005 and 0.03 g/L, between 0.005 and 0.02 g/L, or between 0.005 and 0.01 g/L.

For example, ex-situ produced glucoamylase, preferably as a liquid product, may be dosed in an amount equal to or less than 0.05 grams per one kilo of feed (such as corn slurry), preferably in an amount equal to or less than 0.005 grams per one kilo of feed (for example corn slurry).

Preferably the process of the invention is carried out without adding any glucoamylase. Hence, the dosage of ex-situ produced glucoamylase is preferably zero.

The skilled person knows how to dose glucoamylase. Glucoamylase may be dosed to the fermentation. Glucoamylase can be dosed separately, before or after adding yeast. Glucoamylase can be dosed as a dry product, e.g. as powder or a granulate, or as a liquid. Glucoamylase can be dosed together with other components such as antibiotics. Glucoamylase can also be dosed as part of the back set, i.e. a stream in which part of the thin stillage is recycled e.g. to the fermentation. Glucoamylase can also be dosed using a combination of these methods.

### Redox Sink

Preferably the recombinant yeast cell can further comprise one or more genetic modifications to functionally express a protein that functions in a metabolic pathway forming a non-native redox sink.

For example, these one or more genetic modifications can be one or more genetic modifications for the functional expression of one or more, optionally heterologous, nucleic acid sequences encoding for one or more NAD+/NADH dependent proteins that function in a metabolic pathway to convert NADH to NAD+. Several examples of such metabolic pathways exist, as illustrated further below.

WO2014/081803 describes a recombinant microorganism expressing a heterologous phosphoketolase, phosphotransacetylase or acetate kinase and bifunctional acetaldeyde-alcohol dehydrogenase, incorporated herein by reference; and WO2015/148272 describes a recombinant S. cerevisiae strain expressing a heterologous phosphoketolase, phosphotransacetylase and acetylating acetaldehyde dehydrogenase, incorporated herein by reference. Further WO2018172328A1 describes a recombinant cell that may comprise one or more (heterologous) genes coding for an enzyme having phosphoketolase activity. The phosphoketalase (PKL) routes described in WO2014/081803, WO2015/148272 and WO2018172328A1, provide preferred metabolic pathways to convert NADH to NAD+ and the NADH dependent phosphoketolase described therein is a preferred NADH dependent protein for application in the current invention.

In a preferred embodiment the recombinant yeast cell is therefore a recombinant yeast cell further functionally expressing:
a nucleic acid sequence encoding a protein comprising phosphoketolase activity (EC 4.1.2.9 or EC 4.1.2.22, PKL); and/or
a nucleic acid sequence encoding a protein having phosphotransacetylase (PTA) activity (EC 2.3.1.8); and/or
a nucleic acid sequence encoding a protein having acetate kinase (ACK) activity (EC 2.7.2.12). Preferences for the above proteins and the nucleic sequences encoding for such are as described in WO2014/081803, WO2015/148272 and WO2018172328A1, incorporated herein by reference.

WO2014/129898, WO2018/228836, WO 2018/114762 and WO2019/063542 describe a metabolic route including a protein having ribulose-1,5-biphosphate carboxylase oxygenase (Rubisco) activity, optionally one or more molecular chaperones for a protein having ribulose-1,5-biphosphate carboxylase oxygenase (Rubisco) activity, and a protein having phosphoribulokinase (PRK) activity and recombinant yeast cells comprising such a metabolic route. The metabolic routes described in WO2014/129898, WO2018/228836, WO 2018/114762 and WO2019/063542 are preferred redox sinks and incorporated herein by reference. The genetic modifications and embodiments described for the cell in the claims of WO2014/129898, WO2018/228836, WO 2018/114762 and WO2019/063542, incorporated herein by reference, can advantageously also be present in the recombinant yeast cell of the invention.

In a preferred embodiment the recombinant yeast cell is therefore a recombinant yeast cell further functionally expressing:
a nucleic acid sequence encoding a protein having ribulose-1,5-biphosphate carboxylase oxygenase (Rubisco) activity; and/or
a nucleic acid sequence encoding a protein having phosphoribulokinase (PRK) activity; and/or
optionally a nucleic acid sequence encoding one or more molecular chaperones for the protein having ribulose-1,5-biphosphate carboxylase oxygenase (Rubisco) activity. Preferences for the above proteins and the nucleic sequences encoding for such are as described in WO2014/129898, WO2018/228836 and WO2019/063542.

WO2015/028582 describes examples of a protein comprising NADH dependent acetylating acetaldehyde dehydrogenase activity and metabolic routes incorporating such. The genetic modifications and embodiments described for the cell in the claims of WO2015028582, incorporated herein by reference, can advantageously also be present as a redox sink in the recombinant yeast cell of the invention.

In a preferred embodiment the recombinant yeast cell is therefore a recombinant yeast cell further functionally expressing:
a, preferably heterologous, nucleic acid sequence encoding a protein comprising NADH dependent acetylating acetaldehyde dehydrogenase activity; and/or
a, preferably heterologous, nucleic acid sequence encoding a protein comprising acetyl-CoA synthetase activity; and/or
a, preferably heterologous, nucleic acid sequence encoding a protein comprising alcohol dehydrogenase activity.

Preferences for the above proteins and the nucleic sequences encoding for such are as described in WO2015/028582.

### PPP-Genes

The recombinant yeast cell in the invention may further comprise one or more genetic modifications that increases the flux of the pentose phosphate pathway. The genes encoding for this pentose phosphate pathway are herein also referred to as the “PPP” genes.

In a preferred host cell, the genetic modification comprises overexpression of at least one enzyme of the (non-oxidative part) pentose phosphate pathway. Preferably the enzyme is selected from the group consisting of the enzymes encoding for ribulose-5-phosphate isomerase, ribulose-5-phosphate epimerase, transketolase and transaldolase. Various combinations of enzymes of the (non-oxidative part) pentose phosphate pathway may be overexpressed. E.g. the enzymes that are overexpressed may be at least the enzymes ribulose-5-phosphate isomerase and ribulose-5-phosphate epimerase; or at least the enzymes ribulose-5-phosphate isomerase and transketolase; or at least the enzymes ribulose-5-phosphate isomerase and transaldolase; or at least the enzymes ribulose-5-phosphate epimerase and transketolase; or at least the enzymes ribulose-5-phosphate epimerase and transaldolase; or at least the enzymes transketolase and transaldolase; or at least the enzymes ribulose-5-phosphate epimerase, transketolase and transaldolase; or at least the enzymes ribulose-5-phosphate isomerase, transketolase and transaldolase; or at least the enzymes ribulose-5-phosphate isomerase, ribulose-5-phosphate epimerase, and transaldolase; or at least the enzymes ribulose-5-phosphate isomerase, ribulose-5-phosphate epimerase, and transketolase.

Possibly each of the enzymes ribulose-5-phosphate isomerase, ribulose-5-phosphate epimerase, transketolase and transaldolase are overexpressed in the host cell. More preferred is a host cell in which the genetic modification comprises at least overexpression of both the enzymes transketolase and transaldolase.

### Deletion or Disruption of Glycerol 3-Phosphate Phosphohydrolase And/Or Glycerol 3-Phosphate Dehydrogenase

The recombinant yeast cell further may or may not comprise a deletion or disruption of one or more endogenous nucleotide sequence encoding a glycerol 3-phosphate phosphohydrolase gene and/or encoding a glycerol 3-phosphate dehydrogenase gene.

Preferably enzymatic activity needed for the NADH-dependent glycerol synthesis in the yeast cell is reduced or deleted. The reduction or deletion of the enzymatic activity of glycerol 3-phosphate phosphohydrolase and/or glycerol 3-phosphate dehydrogenase can be achieved by modifying one or more genes encoding a NAD-dependent glycerol 3-phosphate dehydrogenase (GPD) and/or one or more genes encoding a glycerol phosphate phosphatase (GPP), such that the enzyme is expressed considerably less than in the wild-type or such that the gene encodes a polypeptide with reduced activity. Such modifications can be carried out using commonly known biotechnological techniques, and may in particular include one or more knock-out mutations or site-directed mutagenesis of promoter regions or coding regions of the structural genes encoding GPD and/or GPP. Alternatively, yeast strains that are defective in glycerol production may be obtained by random mutagenesis followed by selection of strains with reduced or absent activity of GPD and/or GPP. S. cerevisiae GPD1, GPD2, GPP1 and GPP2 genes are shown in WO2011010923, and are disclosed in SEQ ID NO: 24-27 of that application.

Preferably the recombinant yeast is a recombinant yeast that further comprises a deletion or disruption of a glycerol-3-phosphate dehydrogenase (GPD) gene. The one or more of the glycerol phosphate phosphatase (GPP) genes may or may not be deleted or disrupted.

More preferably the recombinant yeast is a recombinant yeast that comprises a deletion or disruption of a glycerol-3-phosphate dehydrogenase 1 (GPD1) gene. The glycerol-3-phosphate dehydrogenase 2 (GPD2) gene may or may not be deleted or disrupted.

Most preferably the recombinant yeast is a recombinant yeast that comprises a deletion or disruption of a glycerol-3-phosphate dehydrogenase 1 (GPD1) gene, whilst the glycerol-3-phosphate dehydrogenase 2 (GPD2) gene remains active and/or intact. Preferably therefore, only one of the S. cerevisiae GPD1, GPD2, GPP1 and GPP2 genes is disrupted and deleted, whereas most preferably only GPD1 is chosen from the group consisting of GPD1, GPD2, GPP1 and GPP2 genes to be disrupted or deleted.

Without wishing to be bound to any kind of theory it is believed that a recombinant yeast according to the invention wherein the GPD1 gene, but not the GPD2 gene, is deleted or disrupted, can be advantageous when applied in a fermentation process where the glucose at the start of or during the fermentation, is preferably equal to or more than 80 g/L, more preferably equal to or more than 90 g/L, even more preferably equal to or more than 100 g/L, still more preferably equal to or more than 110 g/L, yet even more preferably equal to or more than 120 g/L, equal to or more than 130 g/L, equal to or more than 140 g/L, equal to or more than 150 g/L, equal to or more than 160 g/L, equal to or more than 170 g/L, or equal to or more than 180 g/L.

Preferably at least one gene encoding a GPD and/or at least one gene encoding a GPP is entirely deleted, or at least a part of the gene is deleted that encodes a part of the enzyme that is essential for its activity. Good results can be achieved with a S. cerevisiae cell, wherein the open reading frames of the GPD1 gene and/or of the GPD2 gene have been inactivated. Inactivation of a structural gene (target gene) can be accomplished by a person skilled in the art by synthetically synthesizing or otherwise constructing a DNA fragment consisting of a selectable marker gene flanked by DNA sequences that are identical to sequences that flank the region of the host cell's genome that is to be deleted. Suitably, good results can be been obtained with the inactivation of the GPD1 and GPD2 genes in Saccharomyces cerevisiae by integration of the marker genes kanMX and hphMX4. Subsequently this DNA fragment is transformed into a host cell. Transformed cells that express the dominant marker gene are checked for correct replacement of the region that was designed to be deleted, for example by a diagnostic polymerase chain reaction or Southern hybridization.

Thus, in the recombinant yeast cells of the invention, glycerol 3-phosphate phosphohydrolase activity in the cell and/or glycerol 3-phosphate dehydrogenase activity in the cell can be advantageously reduced.

### Glycerol Re-Uptake

The recombinant yeast cell may or may not further comprise one or more additional nucleic acid sequences that are part of a glycerol re-uptake pathway. That is, the recombinant yeast cell may or may not functionally express:
a nucleic acid sequence encoding for a protein having glycerol dehydrogenase activity (E.C. 1.1.1.6);
a nucleic acid sequence encoding a protein having dihydroxyacetone kinase activity (E.C. 2.7.1.28 or E.C. 2.7.1.29); and
optionally a nucleic acid sequence encoding a protein having glycerol transporter activity.

Without wishing to be bound by any kind of theory it is believed that a recombinant yeast cell that further comprises a combination of glycerol dehydrogenase, dihydroxyacetone kinase and optionally a glycerol transporter has an improved overall performance in the form of higher ethanol yields.

Preferences for the above proteins and the nucleic sequences encoding for such are as described in WO2015/028582 and WO 2018/114762, incorporated herein by reference.

A recombinant yeast cell as described in WO 2018/114762, further incorporating the nucleotide sequences for the glucosidases as described herein is especially preferred.

### Recombinant Expression

The recombinant yeast cell is a recombinant cell. That is to say, a recombinant yeast cell comprises, or is transformed with or is genetically modified with a nucleotide sequence that does not naturally occur in the cell in question. Techniques for the recombinant expression of enzymes in a cell, as well as for the additional genetic modifications of a recombinant yeast cell are well known to those skilled in the art. Typically such techniques involve transformation of a cell with nucleic acid construct comprising the relevant sequence. Such methods are, for example, known from standard handbooks, such as Sambrook and Russel (2001) “Molecular Cloning: A Laboratory Manual”, (3rd edition), published by Cold Spring Harbor Laboratory Press, or F. Ausubel et al., eds., “Current protocols in molecular biology”, Green Publishing and Wiley Interscience, New York (1987). Methods for transformation and genetic modification of fungal host cells are known from e.g. EP-A-0635574, WO98/46772, WO 99/60102, WO00/37671, WO90/14423, EP-A-0481008, EP-A-0635574 and U.S. Pat. No. 6,265,186.

### Fermentation Process

The invention further provides a process for the production of ethanol, comprising converting a carbon source, preferably a carbohydrate or another organic carbon source, using a recombinant yeast cell as described in this specification, thereby forming ethanol.

The feed for this fermentation process suitably comprises one or more fermentable carbon sources. The fermentable carbon source preferably comprises or is consisting of one or more fermentable carbohydrates. More preferably, the fermentable carbon source comprises one or more mono-saccharides, disaccharides and/or polysaccharides. For example, the fermentable carbon source may comprise one or more carbohydrates selected from the group consisting of glucose, fructose, sucrose, maltose, xylose, arabinose, galactose, mannose and trehalose. The fermentable carbon source, preferably comprising or consisting of one or more carbohydrates, may suitably be obtained from starch, cellulose, hemicellulose lignocellulose, and/or pectin. Suitably the fermentable carbon source may be in the form of a, preferably aqueous, slurry, suspension, or a liquid.

The concentration of fermentable carbohydrate, such as for example glucose, during fermentation is preferably equal to or more than 80 g/L. That is, the initial concentration of glucose at the start of the fermentation, is preferably equal to or more than 80 g/L, more preferably equal to or more than 90 g/L, even more preferably equal to or more than 100 g/L, still more preferably equal to or more than 110 g/L, yet even more preferably equal to or more than 120 g/L, equal to or more than 130 g/L, equal to or more than 140 g/L, equal to or more than 150 g/L, equal to or more than 160 g/L, equal to or more than 170 g/L, or equal to or more than 180 g/L. The start of the fermentation may be the moment when the fermentable fermentable carbohydrate is brought into contact with the recombinant cell of the invention.

The fermentable carbon source may be prepared by contacting starch, lignocellulose, and/or pectin with an enzyme composition, wherein one or more mono-saccharides, disaccharides and/or polysaccharides are produced, and wherein the produced mono-saccharides, disaccharides and/or polysaccharides are subsequently fermented to give a fermentation product.

Before enzymatic treatment, the lignocellulosic material may be pretreated. The pretreatment may comprise exposing the lignocellulosic material to an acid, a base, a solvent, heat, a peroxide, ozone, mechanical shredding, grinding, milling or rapid depressurization, or a combination of any two or more thereof. This chemical pretreatment is often combined with heat-pretreatment, e.g. between 150-220° C. for 1 to 30 minutes. Subsequently the pretreated material can be subjected to enzymatic hydrolysis to release sugars that may be fermented according to the invention. This may be executed with conventional methods, e.g. contacting with cellulases, for instance cellobiohydrolase(s), endoglucanase(s), beta-glucosidase(s) and optionally other enzymes, The conversion with the cellulases may be executed at ambient temperatures or at higher temperatures, at a reaction time to release sufficient amounts of sugar(s). The result of the enzymatic hydrolysis is hydrolysis product comprising C5/C6 sugars, herein designated as the sugar composition.

In one embodiment the fermentable carbohydrate is, or is comprised by a biomass hydrolysate, such as a corn stover or corn fiber hydrolysate. Such biomass hydrolysate may in its turn comprise, or be derived from corn stover and/or corn fiber.

By a “hydrolysate” is herein understood a polysaccharide-comprising material (such as corn stover, corn starch, corn fiber, or lignocellulosic material, which polysaccharides have been depolymerized through the addition of water to form mono and oligosaccharide sugars. Hydrolysates may be produced by enzymatic or acid hydrolysis of the polysaccharide-containing material.

A biomass hydrolysate may be a lignocellulosic biomass hydrolysate. Lignocellulose herein includes hemicellulose and hemicellulose parts of biomass. Also lignocellulose includes lignocellulosic fractions of biomass. Suitable lignocellulosic materials may be found in the following list: orchard primings, chaparral, mill waste, urban wood waste, municipal waste, logging waste, forest thinnings, short-rotation woody crops, industrial waste, wheat straw, oat straw, rice straw, barley straw, rye straw, flax straw, soy hulls, rice hulls, rice straw, corn gluten feed, oat hulls, sugar cane, corn stover, corn stalks, corn cobs, corn husks, switch grass, miscanthus, sweet sorghum, canola stems, soybean stems, prairie grass, gamagrass, foxtail; sugar beet pulp, citrus fruit pulp, seed hulls, cellulosic animal wastes, lawn clippings, cotton, seaweed, algae (including macroalgae and microalgae), trees, softwood, hardwood, poplar, pine, shrubs, grasses, wheat, wheat straw, sugar cane bagasse, corn, corn husks, corn hobs, corn kernel, fiber from kernels, products and by-products from wet or dry milling of grains, municipal solid waste, waste paper, yard waste, herbaceous material, agricultural residues, forestry residues, municipal solid waste, waste paper, pulp, paper mill residues, branches, bushes, canes, corn, corn husks, an energy crop, forest, a fruit, a flower, a grain, a grass, a herbaceous crop, a leaf, bark, a needle, a log, a root, a sapling, a shrub, switch grass, a tree, a vegetable, fruit peel, a vine, sugar beet pulp, wheat midlings, oat hulls, hard or soft wood, organic waste material generated from an agricultural process, forestry wood waste, or a combination of any two or more thereof. Algae, such as macroalgae and microalgae have the advantage that they may comprise considerable amounts of sugar alcohols such as sorbitol and/or mannitol. Lignocellulose, which may be considered as a potential renewable feedstock, generally comprises the polysaccharides cellulose (glucans) and hemicelluloses (xylans, heteroxylans and xyloglucans). In addition, some hemicellulose may be present as glucomannans, for example in wood-derived feedstocks. The enzymatic hydrolysis of these polysaccharides to soluble sugars, including both monomers and multimers, for example glucose, cellobiose, xylose, arabinose, galactose, fructose, mannose, rhamnose, ribose, galacturonic acid, glucuronic acid and other hexoses and pentoses occurs under the action of different enzymes acting in concert. In addition, pectins and other pectic substances such as arabinans may make up considerably proportion of the dry mass of typically cell walls from non-woody plant tissues (about a quarter to half of dry mass may be pectins). Lignocellulosic material may be pretreated. The pretreatment may comprise exposing the lignocellulosic material to an acid, a base, a solvent, heat, a peroxide, ozone, mechanical shredding, grinding, milling or rapid depressurization, or a combination of any two or more thereof. This chemical pretreatment is often combined with heat-pretreatment, e.g. between 150-220° C. for 1 to 30 minutes.

The process for the production of ethanol may comprise an aerobic propagation step and an anaerobic fermentation step. More preferably the process according to the invention is a process comprising an aerobic propagation step wherein the population of the recombinant yeast cell is increased; and an anaerobic fermentation step wherein the carbon source is converted to ethanol by using the recombinant yeast cell population.

By propagation is herein understood a process of recombinant yeast cell growth that leads to increase of an initial recombinant yeast cell population. Main purpose of propagation is to increase the population of the recombinant yeast cell using the recombinant yeast cell's natural reproduction capabilities as living organisms. That is, propagation is directed to the production of biomass and is not directed to the production of ethanol. The conditions of propagation may include adequate carbon source, aeration, temperature and nutrient additions. Propagation is an aerobic process, thus the propagation tank must be properly aerated to maintain a certain level of dissolved oxygen. Adequate aeration is commonly achieved by air inductors installed on the piping going into the propagation tank that pull air into the propagation mix as the tank fills and during recirculation. The capacity for the propagation mix to retain dissolved oxygen is a function of the amount of air added and the consistency of the mix, which is why water is often added at a ratio of between 50:50 to 90:10 mash to water. “Thick” propagation mixes (80:20 mash-to-water ratio and higher) often require the addition of compressed air to make up for the lowered capacity for retaining dissolved oxygen. The amount of dissolved oxygen in the propagation mix is also a function of bubble size, so some ethanol plants add air through spargers that produce smaller bubbles compared to air inductors. Along with lower glucose, adequate aeration is important to promote aerobic respiration during propagation, making the environment during propagation different from the anaerobic environment during fermentation.

By an anaerobic fermentation process is herein understood a fermentation step run under anaerobic conditions.

The anaerobic fermentation is preferably run at a temperature that is optimal for the cell. Thus, for most recombinant yeast cells, the fermentation process is performed at a temperature which is less than about 50° C., less than about 42° C., or less than about 38° C. For recombinant yeast cell or filamentous fungal host cells, the fermentation process is preferably performed at a temperature which is lower than about 35, about 33, about 30 or about 28° C. and at a temperature which is higher than about 20, about 22, or about 25° C.

The ethanol yield, based on xylose and/or glucose, in the process according to the invention is preferably at least about 50, about 60, about 70, about 80, about 90, about 95 or about 98%. The ethanol yield is herein defined as a percentage of the theoretical maximum yield.

The process according to the invention, and the propagation step and/or fermentation step suitably comprised therein can be carried out in batch, fed-batch or continuous mode. A separate hydrolysis and fermentation (SHF) process or a simultaneous saccharification and fermentation (SSF) process may also be applied.

For the recovery of the fermentation product existing technologies are used. For different fermentation products different recovery processes are appropriate. Existing methods of recovering ethanol from aqueous mixtures commonly use fractionation and adsorption techniques. For example, a beer still can be used to process a fermented product, which contains ethanol in an aqueous mixture, to produce an enriched ethanol-containing mixture that is then subjected to fractionation (e.g., fractional distillation or other like techniques). Next, the fractions containing the highest concentrations of ethanol can be passed through an adsorber to remove most, if not all, of the remaining water from the ethanol. In an embodiment in addition to the recovery of fermentation product, the yeast may be recycled.

All patent and literature references cited in the present specification are hereby incorporated by reference in their entirety.

### EXAMPLES

The following examples are offered for illustrative purposes only, and are not intended to limit the scope of the present invention in any way.

### General Molecular Biology Techniques

Unless indicated otherwise, the methods used are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

### Starter Strains

Strains were prepared using Ethanol Red® as starting strain. Ethanol Red® is a commercial Saccharomyces cerevisiae strain, available from Lesaffre.

A strain construction approach that can be followed is described in WO2013/144257A1 and WO2015/028582, incorporated herein by reference.

Expression cassettes from various genes of interest can be recombined in vivo into a pathway at a specific locus upon transformation of this yeast (U.S. Pat. No. 9,738,890 B2). The promoter, ORF and terminator sequences are assembled into expression cassettes with Golden Gate technology, as described by Engler et al (2011) and ligated into BsaI-digested backbone vectors that decorated the expression cassettes with the connectors for the in vivo recombination step. The expression cassettes including connectors are amplified by PCR. In addition, a 5′- and a 3′-DNA fragment of the up- and downstream part of the integration locus was amplified using PCR and decorated by a connector sequence. Upon transformation of yeast cells with these DNA fragments, in vivo recombination and integration into the genome takes place at the desired location. CRISPR-Cas9 technology is used to make a unique double stranded break at the integration locus to target the pathway to this specific locus (DiCarlo et al., 2013, Nucleic Acids Res 41:4336-4343) and WO16110512 and US2019309268. The gRNA was expressed from a multi-copy yeast shuttling vector that contains a natMX marker which confers resistance to the yeast cells against the antibiotic substance nourseothricin (NTC). The backbone of this plasmid is based on pRS305 (Sikorski and Hieter, Genetics 1989, vol. 122, pp. 19-27), including a functional 2 micron ORI sequence. The Streptococcus pyogenes CRISPR-associated protein 9 (Cas9) was expressed from a pRS414 plasmid (Sikorski and Hieter, 1989) with kanMX marker which confers resistance to the yeast cells against the antibiotic substance geneticin (G418). The guide RNA and protospacer sequences were designed with a gRNA designer tool (see for example https://www.atum.bio/eCommerce/cas9/input).

For convenience the amino acid substitutions in the glucoamylases as functionally expressed by new strains NX1, NX2, NX3 and NX4, as compared to SEQ ID NO: 1 of the glucoamylase as functionally expressed by comparative strain A are listed in table 3.

TABLE 2
S. cerevisiae strains used in the examples

| Strain name | Genotype |
|---|---|
| Parental strain | Wildtype (Ethanol Red ®) |
| Comparative strain A | PGK1.pro-GA.orf_0048-ENO1.ter |
| Example strain NX4 | PGK1.pro-GA.orf_0034-ENO1.ter |
| Example strain NX3 | PGK1.pro-GA.orf_0033-ENO1.ter |
| Example strain NX2 | PGK1.pro-GA.orf_0010-ENO1.ter |
| Example strain NX1 | PGK1.pro-GA.orf_0009-ENO1.ter |

TABLE 3
The glucoamylase protein (variant polypeptide) functionally epxressed by strains
NX1, NX2, NX3 and NX4 differed from the glucoamylase protein functionally expressed
by comparative strain A in the following amino acid substitutions

| | AA | AA | AA | AA | AA |
|---|---|---|---|---|---|
| Glucoamylase | substitution | substitution | substitution | substitution | substitution |
| Glucoamylase expressed by | V202 | A203 | V333 | Y335 | D336 |
| comparative example A | | | | | |
| Glucoamylase expressed by | | | | Y335M | D336G |
| Example strain NX4 | | | | | |
| Glucoamylase expressed by | | | V333S | Y335M | D336G |
| Example strain NX3 | | | | | |
| Glucoamylase expressed by | V202I | A203N | | Y335M | D336G |
| Example strain NX2 | | | | | |
| Glucoamylase expressed by | V202I | A203N | V333S | Y335M | D336G |
| Example strain NX1 | | | | | |

### Construction of Enzyme Expressing Strains

New enzyme expressing strains were constructed by transforming an S. cerevisiae host cell with enzyme expression cassettes as described below. The S. cerevisiae host cell used in the examples was Ethanol Red®, a S. cerevisiae strain commercially available from Lesaffre.

The genes of interest coding for the enzymes of interest were codon optimized and the native signal sequences were replaced by the S. cerevisiae MATalpha signal sequence: (illustrated by SEQ ID NO: 11).

Synthetic DNA sequences were ordered at TWIST (South San Francisco, CA 94080, USA). or Thermofisher-GeneArt (Regensburg, Germany).

Enzyme expression cassettes were compiled using Golden Gate Cloning and comprised the S. cerevisiae PGK1 promoter (illustrated by SEQ ID NO:12), the gene of interest coding for the enzyme of interest (sequence list SEQ ID NO: 2, 4, 6, 8 and 10 respectively) and the S. cerevisiae ENO1 terminator (illustrated by SEQ ID NO:13).

The cassettes were decorated with 50 bp connectors 2L and 2M to form corresponding constructs. Connector 2L had the nucleotide sequence of SEQ ID NO:14. Connector 2M had the nucleotide sequence of SEQ ID NO:15.

The constructs (each separately to create a separate strain) were integrated at the INT28 locus of the S. cerevisiae host cell, on Chromosome IV between YDR345C (HXT3) and YDRT246C (SVF1) using CRISPR-Cas9 and INT28 protospacer (illustrated by SEQ ID NO:18). Two flanking sequences were used to target homologous integration at INT28: INT28_FLANK5 (illustrated by SEQ ID NO: 16) comprises 100 bp homology with INT28 locus and a unique 50 bp connector “2L” and INT28_FLANK3 (illustrated by SEQ ID NO:17) comprises 100 bp homology with INT28 locus and a unique 50 bp connector “2M”.

Diagnostic PCR was carried out with primers TGGATGTGTGTGCAGTATGCT (SEQ ID NO: 19) and ACAGGAAGTCGAGCGTGTCTGGGT (SEQ ID NO: 20) generating a 414 bp fragment upon correct integration. Constructed strains and their genotypes are listed in table 2.

### Comparative Example A: Construction of Comparative Strain A

Comparative strain A was constructed by transforming reference Ethanol Red® with an expression cassette with the S. cerevisiae PGK1 promoter (see SEQ ID NO: 12), a gene encoding glucoamylase from Punctularia strigosozonata (see SEQ ID NO: 1 and SEQ ID NO: 2, Pstr_GA.orf_0048) as the gene of interest and the S. cerevisiae ENO1 terminator (see SEQ ID NO: 13), and decorated with BsaI sites.

### Example 1: Construction of Example Strain NX1

Example strain NX1 was constructed by transforming reference Ethanol Red® with an expression cassette with the S. cerevisiae PGK1 promoter (see SEQ ID NO: 12), a modified gene (see SEQ ID NO: 03 and SEQ ID NO: 04, GA.orf_0009) as the gene of interest and the S. cerevisiae ENO1 terminator (see SEQ ID NO: 13), and decorated with BsaI sites.

### Example 2: Construction of Example Strain NX2

Example strain NX2 was constructed by transforming reference Ethanol Red® with an expression cassette with the S. cerevisiae PGK1 promoter (see SEQ ID NO: 12), a modified gene (see SEQ ID NO: 05 and SEQ ID NO: 06, GA.orf_0010) as the gene of interest and the S. cerevisiae ENO1 terminator (see SEQ ID NO: 13), and decorated with BsaI sites.

### Example 3: Construction of Example Strain NX3

Example strain NX3 was constructed by transforming reference Ethanol Red® with an expression cassette with the S. cerevisiae PGK1 promoter (see SEQ ID NO: 12), a modified gene (see SEQ ID NO: 07 and SEQ ID NO: 08, GA.orf_0033) as the gene of interest and the S. cerevisiae ENO1 terminator (see SEQ ID NO: 13), and decorated with BsaI sites.

### Example 4: Construction of Example Strain NX4

Example strain NX4 was constructed by transforming reference Ethanol Red® with an expression cassette with the S. cerevisiae PGK1 promoter (see SEQ ID NO: 12), a modified gene (see SEQ ID NO: 09 and SEQ ID NO: 10, GA.orf_0034) as the gene of interest and the S. cerevisiae ENO1 terminator (see SEQ ID NO: 13), and decorated with BsaI sites.

### Example 5: Fermentations With Comparative Strains A, and New Strains NX1, NX2, NX3 and NX4

Precultures of comparative strain A and new strains NX1, NX2, NX3, NX4 were made as follows: Glycerol stocks (−80° C.) were thawed at room temperature and used to inoculate 0.2 L mineral medium (as described by Luttik, M L H. et al (2000) in their article titled “The Saccharomyces cerevisiae ICL2 Gene Encodes a Mitochondrial 2-Methylisocitrate Lyase Involved in Propionyl-Coenzyme A Metabolism”, published in J. Bacteriol. Vol. 182, pages 7007-7013) supplemented with 2% (w/v) glucose, at pH 6.0 (adjusted with 2M H2SO4/4N KOH), in non-baffled 0.5 L shake-flasks. The precultures were incubated for 16 to 20 hours at 32° C. and shaken at 200 RPM. After determination of the yeast cell dry weight (CDW) through OD600 measurement (using an existing CDW vs OD600 calibration line), a quantity of preculture corresponding to the required 0.5 g CDW/liter inoculum concentration for the propagation was centrifuged (3 min, 5300×g), washed once with one sample volume sterile demineralized water, centrifuged once more, and resuspended in propagation medium.

Propagation of comparative strain A and new strains NX1, NX2, NX3, NX4 was carried out as follows: A propagation step was performed in 100 mL non-baffled shake flasks, using 20 mL diluted corn mash (70% v/v Com mash: 30% v/v demineralized water) supplemented with 1.25 g/liter(L) urea (as nitrogen source) and an antibiotic mix (comprising 1 ml 100 μg/L penicillin G & 1 ml 50 μg/L Neomycin stock per liter of corn mash). After all additions, the pH was adjusted to 5.0 using 4N KOH/2M H2SO4. All strains were inoculated at 0.5 g CDW/L as described above and propagations for all strains were ran for 6 hrs at 32° C. shaking at 140 RPM. During propagation external (ex-situ generated) glucoamylase (Spirizyme, commercially obtainable from Novozymes) was dosed at a dosage of 0.1 g/kg (i.e. 0.1 mL/L).

Main fermentations of comparative strain A and new strains NX1, NX2, NX3, NX4 were carried out as follows: A main fermentation step was performed using 200 ml medium in 500 ml Schott bottles equipped with pressure recording/releasing caps (Ankom Technology, Macedon NY, USA), while shaking at 140 rpm and 32° C. pH was not controlled during fermentation. Fermentations were stopped after 66 h. Fermentations were executed with corn mash having dry solids (DS) content of about 33.4% w/w. Subsequently, the com mash was supplemented with 1 g/L urea, and the antibiotics: neomycin and penicillin G to a final concentration of 50 μg/mL and 100 g/mL (i.e. adding solutions 100 mg/ml PenG stock+50 mg/ml Neomycin stock respectively); antifoam (Basildon, approximately 0.5 mL/L). After all additions, the pH was adjusted to 5.0 using 2M H2SO4/4N KOH. The required yeast pitch from propagation to fermentation was 1.5% on fermentation volume. During the main fermentation external (ex-situ generated) glucoamylase (Spirizyme, commercially obtainable from Novozymes) was dosed at 0.24 g/kg (i.e. 0.24 mL/L).

Sampling of the fermentation was carried out as follows: The end of fermentation was at 66 hours. Since the fermentation broths contained active glucoamylase enzyme, 50 μl of a 10 g/L acarbose stock solution was added to approximately 5 g sample to stop glucoamylase activity. Samples were taken at the end of fermentation (at 66 hours) to assess effects of the expressed enzyme activities on sugar release profiles throughout the fermentation. Samples for HPLC analysis were separated from yeast biomass and insoluble components (com mash) by passing the clear supernatant after centrifugation through a 0.2 μm pore size filter. HPLC (Aminex) analysis was conducted. The results are listed in table 4.

Conclusions were as follows: Residual sugars (mg/L) at the end of fermentation (66 hours) were measured by HPLC. The results are summarized in Table 4 below. It was found that the example strains NX1, NX2, NX3 and NX4 convert more maltose and that the remaining amount of total residual sugars at the end of fermentation was similar or less than that obtained for the comparative strain A.

TABLE 4
Residual sugars at the end of fermentation
(66 hours) measured by HPLC (mg/L)

| | Maltose | Sum of total |
|---|---|---|
| Strain | (mg/liter) | residual sugars |
| Comparative strain A | 1031 | 6220 |
| [P. strigosozonata glucoamylase] | | |
| Example strain NX4 | 724 | 6506 |
| Example strain NX3 | 638 | 5954 |
| Example strain NX2 | 695 | 5625 |
| Example strain NX1 | 572 | 5120 |

1. A variant polypeptide of a parent polypeptide,
wherein the parent polypeptide comprises the amino acid sequence of SEQ ID NO: 1, and
wherein the variant polypeptide comprises an amino acid sequence which, when aligned with the amino acid sequence of SEQ ID NO: 1, comprises an amino acid substitution of V202I and/or A203N and/or V333S and/or Y335M and/or D336G, the positions of said amino acids being defined with reference to the amino acid sequence of SEQ ID NO: 1.

2. The variant polypeptide according to claim 1, wherein the variant polypeptide comprises or consists of an amino acid sequence having equal to or more than 70%, and more preferably equal to or more than 75%, 80%, 85%, 90%, 95, 98%, or 99%, sequence identity with the amino acid sequence of SEQ ID NO: 01.

3. The variant polypeptide according to claim 1 or 2, wherein any further amino acid substitutions in the variant polypeptide as compared to the parent polypeptide of SEQ ID NO: 1, other than Y335M and/or D336G and/or V202I and/or A203N and/or V333S, are conservative amino acid substitutions.

4. The variant polypeptide according to any one of claims 1 to 3, wherein the variant polypeptide is a polypeptide comprising or consisting of an amino acid sequence of SEQ ID NO:03, SEQ ID NO:05, SEQ ID NO: 07 or SEQ ID NO:09 or an amino acid sequence having equal to or more than 70%, and more preferably equal to or more than 75%, 80%, 85%, 90%, 95, 98%, or 99%, sequence identity with the amino acid sequence of SEQ ID NO:03, SEQ ID NO:05, SEQ ID NO: 07 and/or SEQ ID NO:09.

5. The variant polypeptide according to any one of claims 1 to 4, wherein the variant polypeptide is a protein having glucoamylase activity.

6. The variant polypeptide according to any one of claims 1 to 5, wherein the variant polypeptide is synthetic polypeptide.

7. A nucleotide sequence comprising or consisting of nucleotide sequence of respectively SEQ ID NO: 04, SEQ ID NO:06, SEQ ID NO: 08 or SEQ ID NO: 10.

8. A recombinant yeast cell functionally expressing a nucleotide sequence encoding a variant polypeptide according to any one of claims 1 to 6, or a nucleotide sequence of claim 7.

9. A recombinant yeast cell comprising or functionally expressing a nucleotide sequence encoding a polypeptide, which polypeptide comprises or consists of an amino acid sequence of SEQ ID NO:03, SEQ ID NO:05, SEQ ID NO: 07 or SEQ ID NO:09 or an amino acid sequence having equal to or more than 70%, and more preferably equal to or more than 75%, 80%, 85%, 90%, 95, 98%, or 99%, sequence identity with the amino acid sequence of SEQ ID NO:03, SEQ ID NO:05, SEQ ID NO: 07 and/or SEQ ID NO: 09.

10. The recombinant yeast cell according to claim 9, wherein the polypeptide comprises
an amino acid sequence of SEQ ID NO: 03, SEQ ID NO: 05, SEQ ID NO: 07 or SEQ ID NO: 09; or
an amino acid sequence which has at least 70% sequence identity, preferably at least 75%, 80%, 85%, 90%, 95, 98%, or 99% sequence identity with the amino acid sequence of SEQ ID NO: 03, SEQ ID NO: 05, SEQ ID NO: 07 and/or SEQ ID NO: 09 and preferably comprises no alterations in the amino acid at location 202, 203, 333, 335 or 336.

11. The recombinant yeast cell according to any one of claims 8 to 10, comprising or functionally expressing a nucleotide sequence comprising or consisting of nucleotide sequence SEQ ID NO:04, SEQ ID NO:06, SEQ ID NO: 08 or SEQ ID NO:10 or a nucleotide sequence having equal to or more than 70%, and more preferably equal to or more than 75%, 80%, 85%, 90%, 95, 98%, or 99%, sequence identity with the nucleotide sequence of SEQ ID NO:04, SEQ ID NO:06, SEQ ID NO: 08 or SEQ ID NO: 10

12. The recombinant yeast cell according to any one of claims 8 to 11, wherein the polypeptide is a protein having glucoamylase activity.

13. The recombinant yeast cell according to any one of claims 8 to 12, wherein the recombinant yeast cell is a recombinant Saccharomyces yeast cell, more preferably a recombinant Saccharomyces cerevisiae yeast cell.

14. A process for the production of ethanol, comprising converting a carbon source, preferably a carbohydrate, using a variant polypeptide according to any one of claims 1 to 6, or a recombinant yeast cell according to any one of claims 8 to 13.

15. The process according to claim 14, wherein the process comprises external dosing of a glucoamylase at a concentration of 0.05 g/L or less, expressed as the total amount of glucoamylase enzyme in grams per liter of carbon source comprising feed.

16. The process according to claim 14, wherein the process is carried out without external dosing of any glucoamylase.
